(19) United States
(12) Patent Application Publication
(10) Pub. No.: US 2020/0297310 A1
(43) Pub. Date: Sep. 24, 2020

(54) MEDICAL TOILET WITH ACOUSTIC TRANSDUCERS FOR COLLECTING HEALTH-RELATED MEASUREMENTS

(71) Applicants: David R. Hall, Provo, UT (US); K. Jeffrey Campbell, Spanish Fork, UT (US); Joshua Larsen, Spanish Fork, UT (US); Jared Reynolds, Pleasant Grove, UT (US); Daniel Hendricks, Provo, UT (US); Travis Niederhauser, Mapleton, UT (US); Steven J.M. Butala, Provo, UT (US); Joshua D. Heiner, Lehi, UT (US); A. Terrece Pearman, Draper, UT (US); Vivek Garg, Sandy, UT (US)

(72) Inventors: David R. Hall, Provo, UT (US); K. Jeffrey Campbell, Spanish Fork, UT (US); Joshua Larsen, Spanish Fork, UT (US); Jared Reynolds, Pleasant Grove, UT (US); Daniel Hendricks, Provo, UT (US); Travis Niederhauser, Mapleton, UT (US); Steven J.M. Butala, Provo, UT (US); Joshua D. Heiner, Lehi, UT (US); A. Terrece Pearman, Draper, UT (US); Vivek Garg, Sandy, UT (US)

(21) Appl. No.: 16/360,708

(22) Filed: Mar. 21, 2019

(51) Int. Cl. A61B 8/00; A61B 5/00

(52) U.S. Cl. CPC A61B 8/4272; A61B 5/6823; A61B 8/54; E03D 5/00; A61B 5/6831; A61B 5/4509; A61B 5/6891; A61B 8/4227

(57) ABSTRACT

The medical toilet may include a seat, lid, and bowl as does a traditional toilet. It also includes one or more acoustic transducers. The acoustic transducers may be located on the seat where they may measure bone density in a user's pelvis or femur. The acoustic transducers may be located on the lid or on a belt which wraps around the user. These acoustic transducers may collect data relating to a user's heart, lungs, liver, bowel, or other internal organs. The medical toilet may include a handrail with an acoustic transducer which measures the bone density in the user's wrist, radius, or ulna. A controller associated with the medical toilet may store algorithms for analyzing the data collected by the acoustic transducers. The controller may perform machine learning to improve the analyses and may calculate trends in repeated measurements taken from the same user or a population of users.

### BACKGROUND

### Field of the Invention

This disclosure relates to diagnostic devices which use acoustic transducers including echocardiogram and other ultrasound techniques.

### Background of the Invention

It is medically advantageous to non-invasively monitor the breathing and heart function of a human. Medical professionals routinely use sound-enhancing instruments, including echocardiogram and ultrasound devices, to detect abnormalities in patient organs. Frequent analysis and monitoring of physiological functions, for example, daily or multiple times per day, would provide additional benefit, including the acquisition of mass data suitable for machine learning applications. Typically, monitoring by a health professional at this frequency is too costly and cumbersome to be feasible. However, people use a toilet multiple times each day. A toilet which could collect measurements related to the user's health at each use could accumulate numerous and frequent measurements and track changes in the measurements over time.

### BRIEF SUMMARY OF THE INVENTION

The present disclosure describes a medical toilet which implements a sonic/acoustic transducer in the seat, lid, bowl, or other locations within a modern toilet. The medical toilet uses the acoustic transducer to capture sound waves emitted through a human body and includes a controller which analyzes the sound waves. Algorithms stored on the controller may confirm a user's health or identify abnormalities which are relevant to the user's health and wellness. The analyses may include, but is not limited to, breathing and heart function.

Like a traditional toilet, the disclosed medical toilet may include a toilet seat, toilet lid, and toilet bowl. However, unlike a traditional toilet, the medical toilet may also include one or more acoustic transducers and a controller. The controller may be disposed within the toilet or it may be a remote device to which the toilet transmits data through wired or wireless techniques. In some embodiments, the one or more acoustic transducers may be located on a belt which may be wrapped around a user's chest or torso. One or more acoustic transducers may be disposed on a hand-held probe which a user may direct toward a desired body part.

One or more acoustic transducers may be directed toward the toilet bowl. In an example, the one or more acoustic transducers may be disposed on the lower side of the toilet seat. The acoustic transducer may detect sound waves created by human waste contacting the surface of the toilet water. The distance between the surface of the toilet water and the acoustic transducer may be calculated to determine the volume of human waste added and the rate the waste is deposited into the toilet bowl.

Some acoustic transducers may emit Doppler signals, either continuous or pulsatile. The signals may enable the medical toilet to conduct Doppler ultrasound testing.

The controller may store algorithms which may analyze the signals the acoustic transducers collect. The algorithms may create two-, three-, or four-dimensional images of a user's vital organs which may be viewed on a computer screen. In an example, the algorithms may then analyze the images to identify cardiomyopathies, estimate bone density, or other data relevant to a user's health and wellness. The algorithms may conduct machine learning to improve the accuracy and/or consistency of the data analysis. The algorithms may calculate trends in repeated measurements collected from the same user or a population of users. These repeated measurements may be used to train the controller to perform machine learning.

The controller may be within or local to the medical toilet or it may be a remote device. The controller within or local to the medical toilet may store algorithms which direct the medical toilet to transmit data to a remote device through wired or wireless methods. A healthcare provider may analyze the data stored on the remote device. Data from multiple medical toilets may be analyzed to train the controller to improve machine learning and to calculate trends amongst a population of users.

### BRIEF DESCRIPTION OF THE DRAWINGS

FIG. 1 is a perspective view of a medical toilet with a single acoustic transducer according to an embodiment of the disclosure.

FIG. 2 is a perspective view of a medical toilet with a plurality of acoustic transducers according to an embodiment of the disclosure.

FIG. 3 is a perspective view of a medical toilet with a matrix array of acoustic transducers according to an embodiment of the disclosure.

FIG. 4 is a cross sectional view of a medical toilet with an acoustic transducer beneath the toilet seat receiving signals from the toilet water surface.

FIG. 5 is a perspective view of a user seated on a medical toilet according to an embodiment of the disclosure and using a hand-held probe.

FIG. 6 is a schematic view of a medical toilet according to an embodiment of the disclosure sending data wirelessly to a remote device.

FIG. 7 is a perspective view of a medical toilet with an armrest which includes an acoustic transducer according to an embodiment of the disclosure.

### DETAILED DESCRIPTION OF THE INVENTION

Definitions:

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure, and are not meant to be limiting in any fashion. Nor do these phrases indicate any kind of preference for the disclosed embodiment.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

We disclose a medical toilet which a user may sit on and deposit bodily waste therein as is typically done with a traditional toilet. The medical toilet may include a toilet seat, a toilet lid, and a toilet bowl. In addition, the disclosed medical toilet may include one or a plurality of acoustic transducers. In some embodiments which include a plurality of acoustic transducers, the acoustic transducers are positioned in a matrix array. This configuration may enable simultaneous or sequential collection of different types of acoustic signals and/or simultaneously collection of signals from different organs. For example the matrix array may simultaneously or sequentially collect acoustic signals from a user's heart and lungs. The medical toilet may also include a controller on which non-transitory computer readable medium may store collected acoustic data as well as algorithms for performing data analysis as disclosed herein.

The one or more acoustic transducers may be disposed on one or more parts of the medical toilet. For example, one or more acoustic transducers may be disposed on a toilet seat of the medical toilet. In this example, ultrasound data may measure a user's bone density in the femur shaft or greater trochanter.

In another embodiment, the one or more acoustic transducers may be disposed on a toilet lid on the medical toilet. The acoustic transducers may collect echocardiogram data or data which is indicative of the health of the user's lungs.

Some embodiments of the medical toilet may include an arm rest. A user may place an arm on the arm rest and one or more acoustic transducers disposed on the arm rest may collect ultrasound data, for example, data relevant for determining a user's bone density in the wrist or radius and ulna.

Some embodiments of the disclosed medical toilet include a sensor platform array platform as disclosed in U.S. patent application Ser. No. 16/272,206 filed on Feb. 11, 2019 which is hereby incorporated by reference in its entirety. An acoustic transducer may be disposed on one or more of the sensor platforms, each of which may be moved to interact with the user at an optimal location on the user's body.

In another embodiment, at least one of the acoustic transducers is disposed on a hand-held probe. A user may hold the hand-held probe in one or the other of the user's hands and direct the probe toward a body part or organ which the user wishes to monitor.

In some embodiments, at least one of the at least one acoustic transducer is directed toward the toilet bowl in the medical toilet. In an example, an acoustic transducer is disposed on a lower side of the toilet seat. The acoustic transducer may emit a soundwave toward the surface of the toilet bowl water which bounces off the surface of the water and back to the acoustic transducer. An algorithm stored on the controller may determine the distance between the acoustic transducer and the surface of the water based on the angle and/or time at which the sound wave returns to the acoustic transducer. The algorithm may use this measurement to calculate the toilet water volume prior to the addition of waste.

As a user deposits urine or feces into the toilet bowl, the acoustic transducer may detect soundwaves created when the waste strikes the surface of the toilet bowl water. Algorithms on the controller within the medical toilet may calculate the duration of a urination or defecation event and differentiate between urination or defecation due to the sound pattern. An additional sound wave may then be emitted to assess the increased volume in the toilet bowl by determining the change in the distance between the surface of the toilet water and the acoustic transducer. In this way, the increased level of the toilet water may be determined which is proportional to the volume of waste added to the toilet water. Accordingly, the urine volume or feces volume may be calculated. In some embodiments, the acoustic transducer may collect continuous measurements as the user voids into the medical toilet which may result in calculated urine flow rate or rate of defecation. In some embodiments, the acoustic transducer may detect sounds made from flatulence and the analysis stored in the controller.

In some embodiments, the medical toilet includes a belt which may be wrapped across a user's chest or abdomen. The belt may include one or more acoustic transducers on its inner surface which places them against the user's body when the belt is wrapped around the user. The acoustic transducers may collect readings from the user's internal organs as collected from the ventral side of the user's body including heart, lung and bowel sounds.

In some embodiments, the one or more acoustic transducers emits Doppler signals resulting in a Doppler ultrasound. The Doppler signals may be either pulsed or continuous. This embodiment may be used to estimate the blood flow through blood vessels. Doppler ultrasound may identify blood clots, poorly functioning heart and vein valves, peripheral artery disease, aneurisms, and arterial stenosis.

The controller within or in electronic connection with the medical toilet may include non-transitory computer readable medium which may include algorithms for processing the data the one or more acoustic transducer collects. The controller may be local (within or connected by electrical wiring to the medical toilet) or remote and in electronic communication with the medical toilet. In an example, the algorithms may create two-, three-, or four-dimensional images based on the signals the one or more acoustic transducer collects. In an example, the algorithm may create a two-dimensional, still image of a user's heart, a three-dimensional image of the user's heart, or, by adding time as the fourth dimension, a moving image of the heart as it contracts. These images may be interpreted by a medical professional to diagnose cardiomyopathies. Alternatively, the algorithms stored on the controller may identify a cardiomyopathy, bone density, pneumonia, and other pathologies.

The medical toilet may collect repeated measurements from the same user over a period of time. The algorithms stored on the controller may calculate averages, means, and trends from the repeated measurements. Machine learning may improve the accuracy and/or consistency of the data collected. In order to perform machine learning, the controller may be trained either on data from a single medical toilet which collects data from one or more user or from data which a plurality of medical toilets transmit to a remote controller. Accordingly, the controller on the medical toilet may store instructions for transmitting each measurement and/or each calculated trend to a remote device. In an example, the remote device may be a cloud database. In another example, the remote device may be a computer in a hospital, clinic, or other healthcare facility. A healthcare provider may access the user's data for diagnostic purposes or to suggest changes to the user's lifestyle which would promote the user's health and wellness.

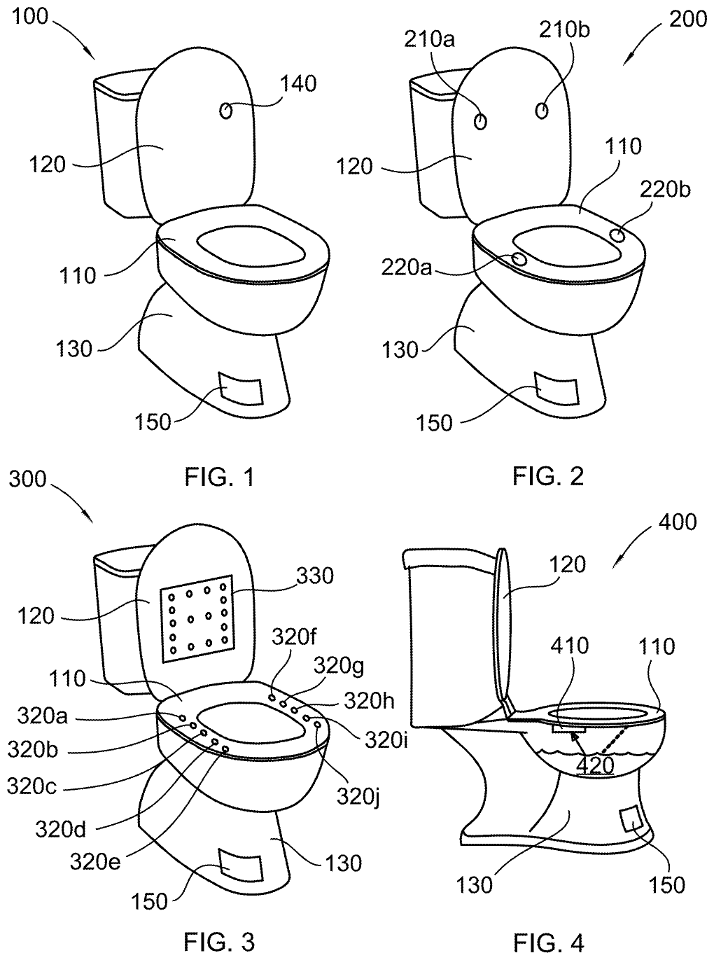

Referring now to the drawings, FIG. 1 illustrates medical toilet 100 which is an embodiment of the disclosed medical toilet. Medical toilet 100 includes toilet seat 110, toilet lid 120, and base 130 as is typical of a traditional western style toilet. In addition, toilet 100 includes acoustic transducer 140 which, in this embodiment, is disposed on toilet lid 120. In this embodiment, acoustic transducer 140 is positioned on the left side of toilet lid 120 which would be near a user's heart and left lung when the user is seated on medical toilet 100. A user may be seated on medical toilet 100 and lean back against toilet lid 120. Acoustic transducer 140 may contact the user's back and collect acoustic data which may indicate the function of the user's heart, lungs, vertebral bone density, or combinations thereof. The acoustic signals may be stored and analyzed on controller 150 which, in this embodiment, is housed within base130.

FIG. 2 illustrates medical toilet 200 which is another embodiment of the disclosed medical toilet. Like medical toilet 100, medical toilet 200 includes toilet seat 110, toilet lid 120, and base 130 as is typical of a traditional western style toilet. In contrast with the single acoustic transducer on medical toilet 100, medical toilet 200 includes acoustic transducers 210a and 210b disposed on toilet lid 120 and acoustic transducers 220a and 220b on toilet seat 110.

A user may be seated on medical toilet 200 with toilet lid 120 against the user's back. When seated on medical toilet 200, acoustic transducers 210a and 210b are positioned approximately behind a user's lungs. Acoustic transducer 210b is positioned behind the user's heart. Consequently, when the acoustic signals are transmitted to controller 150, data which may indicate the health of the user's heart and lungs may be created using algorithms stored within controller 150. Other measurements, for example, vertebral bone density may also be assessed using the acoustic signals collected from acoustic transducers 210a and 210b.

Acoustic transducers 220a and 220b are in close proximity to the femur shaft and greater trochanter when a user is seated on medical toilet 200. Acoustic transducers 220a and 220b may collect ultrasound data which may be used to provide an assessment of a user's bone density in the femur and/or pelvis.

FIG. 3 illustrates medical toilet 300 which is another embodiment of the disclosed medical toilet. Like medical toilets 100 and 200, medical toilet 300 includes toilet seat 110, toilet lid 120, and base 130 as is typical of a traditional western style toilet. Toilet 300 includes acoustic transducers 320a-j disposed on toilet seat 110 which are near the shaft and greater trochanter of a user's femur when the user is seated on toilet seat 110. Acoustic transducers 320a-j may collect ultrasound data all along the user's femur and/or pelvis. The ultrasound data may be transmitted to controller 150 which may store algorithms which assess the user's bone density based on the ultrasound data.

Matrix array 330 is disposed on toilet lid 120. Matrix array 330 comprises a plurality of acoustic transducers which come in contact with a seated user's back when the user leans against toilet lid 120. Data indicating a user's bone density in the vertebrae may be collected using ultrasound, echocardiogram data may be collected, and sounds waves indicating whether a user's lungs are healthy may be collected through the acoustic transducers within matrix array 330.

FIG. 4 is a cross sectional view of medical toilet 400. Like medical toilets 100, 200, and 300, medical toilet 400 includes toilet seat 110, toilet lid 120, and base 130 as is typical of a traditional western style toilet. Additionally, medical toilet 400 includes acoustic transducer 410 which is disposed on the underside of toilet seat 110. When a user urinates or defecates into medical toilet 400, acoustic transducer 410 receives the sound created from the waste material colliding with the surface of toilet water 420 as is indicated by the solid arrow. The distance between the surface of toilet water 420 and the acoustic transducer may be calculated using algorithms stored on controller 150. Accordingly, urine volume and flow rate may be determined as a user urinates. Volume of feces deposited into toilet water 420 may also be determined.

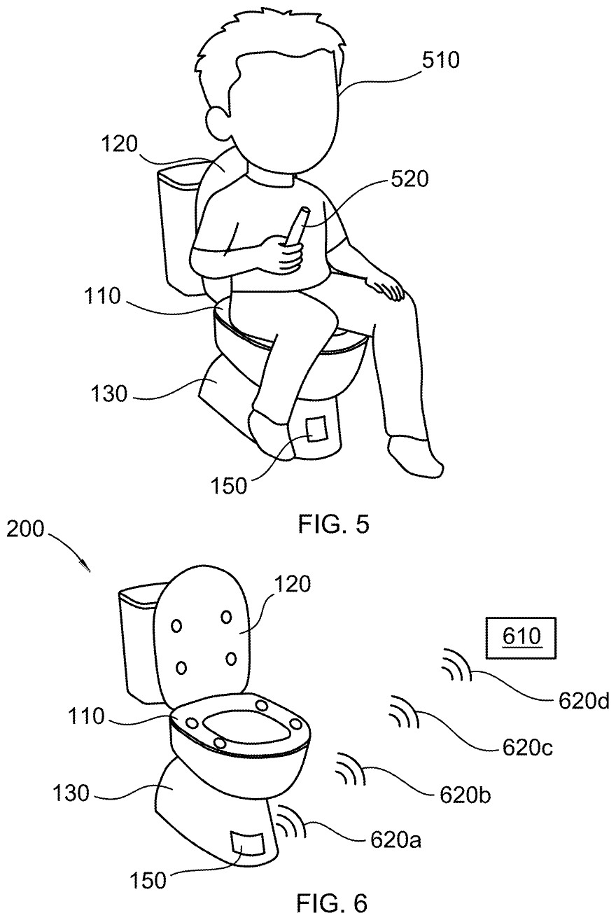

FIG. 5 illustrates user 510 seated on an embodiment of the disclosed medical toilet which includes hand-held probe 520. An acoustic transducer may be disposed on hand-held probe 520. User 510 is shown moving hand-held probe 520 toward his chest near his heart. User 510 may position hand-held probe 520 toward other body parts to collect data relating to other organs.

FIG. 6 illustrates medical toilet 200, first presented in FIG. 2, as it transmits data wirelessly from controller 150 to remote device 610. The transmission is illustrated with wireless signals 620a-d. Remote device 610 may be a cloud database, a computer in a hospital, clinic, or other healthcare facility, or any other remote electronic storage or computing device. A healthcare provider may access the user's data on remote device 610 to assess the user's health and wellness status.

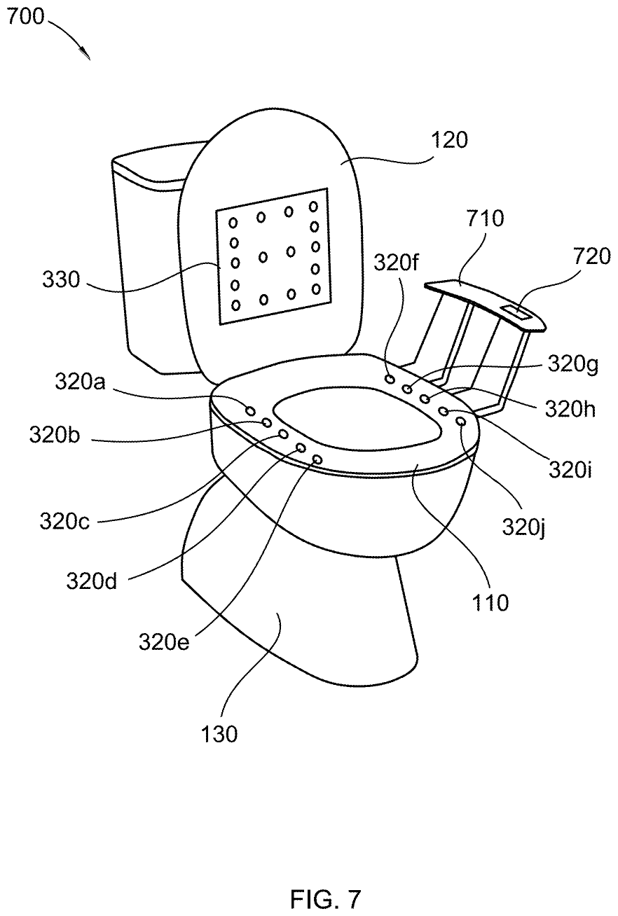

FIG. 7 illustrates medical toilet 700 which is similar to medical toilet 300 first presented in FIG. 3. However, medical toilet 700 includes hand rail 710. Acoustic transducer 720 is disposed on hand rail 710. A user may place an arm on hand rail 710 with a wrist adjacent to acoustic transducer 720. Acoustic transducer 720 may collect ultrasound data to indicate the user's bone density in the user's wrist, radius, and/or ulna.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A medical toilet comprising:
a. a toilet seat;
b. a toilet lid;
c. a toilet bowl;
d. at least one acoustic transducer disposed on one or more of the following list: the toilet seat, the toilet lid, and the toilet bowl; and
e. a controller.

2. The medical toilet of claim 1, wherein at least one acoustic transducer comprises a plurality of acoustic transducers.

3. The medical toilet of claim 2, wherein the plurality of acoustic transducers comprises a matrix array.

4. The medical toilet of claim 3, wherein the matrix array is disposed on the toilet lid.

5. The medical toilet of claim 3, wherein the controller comprises a non-transitory computer readable medium which stores instructions for creating a two-dimensional image based on a series of acoustic signals detected by the plurality of acoustic transducers.

6. The medical toilet of claim 3, wherein the controller comprises a non-transitory computer readable medium which stores instructions for creating a three-dimensional image based on a series of acoustic signals detected by the plurality of acoustic transducers.

7. The medical toilet of claim 3, wherein the controller comprises a non-transitory computer readable medium which stores instructions for creating a four-dimensional image based on a series of acoustic signals detected by the plurality of acoustic transducers.

8. The medical toilet of claim 3, wherein the plurality of acoustic transducers is disposed on at least one sensor platform array.

9. The medical toilet of claim 1, wherein at least one of the at least one acoustic transducers is disposed on the toilet seat.

10. The medical toilet of claim 1, wherein at least one of the at least one acoustic transducers is disposed on the toilet lid.

11. The medical toilet of claim 1, wherein at least one of the at least one acoustic transducers is directed towards the toilet bowl.

12. The medical toilet of claim 11, wherein the controller comprises non-transitory computer readable medium which stores instructions for identifying one or more of the following list based on a series of acoustic signals detected by the at least one acoustic transducer: a length of urination time, urine volume, consistency of urine flow, flatulence, defecation, and volume of solid waste.

13. The medical toilet of claim 1 further comprising a belt, wherein at least one of the at least one acoustic transducers is disposed on an inner surface of the belt.

14. The medical toilet of claim 1, wherein at least one of the at least one acoustic transducers emits Doppler signals.

15. The medical toilet of claim 1, further comprising an arm rest, wherein at least one of the at least one acoustic transducers is disposed on the arm rest.

16. The medical toilet of claim 1, further comprising a hand-held probe; wherein at least one of the at least one acoustic transducer is disposed on the hand-held probe.

17. The medical toilet of claim 1, wherein the controller comprises non-transitory computer readable medium which stores instructions for identifying a cardiomyopathy.

18. The medical toilet of claim 1, wherein the controller comprises non-transitory computer readable medium which stores instructions for determining bone density.

19. The medical toilet of claim 1, wherein the controller comprises non-transitory computer readable medium which stores instructions for calculating trends in measurements collected by the at least one acoustic transducer.

20. The medical toilet of claim 19, wherein the controller comprises non-transitory computer readable medium which stores instructions for transmitting the trends in measurements to a remote device.
